(19) United States
(12) Patent Application Publication
(10) Pub. No.: US 2008/0312553 A1
(43) Pub. Date: Dec. 18, 2008

(54) INTRACORPOREAL PRESSURE MEASUREMENT DEVICES AND METHODS

(76) Inventor: Michael J. Timmons, Lake Elmo, MN (US)

Correspondence Address: FAEGRE & BENSON, LLP;32469, 2200 WELLS FARGO CENTER, 90 SOUTH SEVENTH STREET, MINNEAPOLIS, MN, 55402-3901, US

(21) Appl. No.: 12/125,700

(22) Filed: May 22, 2008

Related U.S. Application Data
(60) Provisional application No. 60/943,944, filed on Jun. 14, 2007.

(51) Int. Cl. A61B 5/03

(52) U.S. Cl. 600/561

(57) ABSTRACT

The invention relates to devices, systems, and methods for the measurement of a pressure within a body that is adjusted to compensate for variations in local atmospheric pressure. A pressure measurement system can include an implantable target pressure sensor, an implantable internal reference pressure sensor located remotely from the target pressure sensor, an external reference pressure sensor configured to transmit a telemetric signal that is indicative of the local atmospheric pressure, and at least one condition indicator. The implantable medical device system further includes a controller configured to determine a correlation factor based on a signal from the implantable reference pressure sensor and the signal from the external reference pressure sensor.

### CROSS-REFERENCE TO RELATED APPLICATION

This application claims priority under 35 U.S.C. § 119 to U.S. Provisional Application No. 60/943,944, filed Jun. 14, 2007, entitled “Intracorporeal Pressure Measurement Devices and Methods,” which is herein incorporated by reference in its entirety.

### TECHNICAL FIELD

The invention relates to the measurement of a pressure within the body of a living being. More particularly, the invention relates to devices and methods for the measurement of a pressure within a body that is adjusted to compensate for variations in local atmospheric pressure.

### BACKGROUND

A variety of implantable medical devices are used to provide medical therapy to patients. One example of an implantable medical device is a cardiac rhythm management (CRM) device. Examples of CRM devices include pacemakers and implantable cardioverter defibrillators (ICD). These devices provide medical treatment to patients having disorders relating to heart rhythm, such as bradycardia or tachycardia. For example, a patient with bradycardia may be fitted with a pacemaker configured to monitor the patient's heart rate and to provide an electrical pacing pulse to the cardiac tissue. By way of further example, a patient may have an ICD implanted to provide an electrical shock to the patient's heart if the patient experiences fibrillation.

In certain patients having implantable medical devices, it can be desirable to measure a physiological pressure within the body. As a specific example, in some patients it may be desirable to measure an intracardiac pressure, such as a ventricular pressure. In some implantable medical devices, for example, the measuring and monitoring of an intracardiac pressure may permit more accurate control of the therapy provided by the devices. In addition, in some cases, measuring a pressure such as an intracardiac pressure can provide data to aid in the diagnosis of various medical conditions.

However, in patients where a physiological pressure is to be measured, the implantable medical device typically only provides an absolute pressure measurement, the sum of both a physiological pressure component and an atmospheric pressure component. For purposes of controlling the therapy provided by an implantable medical device, it is desirable to be able to accurately determine changes in the physiological pressure component alone, without changes in atmospheric pressure affecting the physiological pressure readings. This is because small but significant changes in physiological pressure can be masked, mimicked, or distorted by changes in local atmospheric pressure. For example, a change in elevation of forty feet can result in an atmospheric pressure change of approximately 1 mmHg, depending on the starting elevation. As a result, riding in an elevator or riding in an airplane can result in significant local atmospheric pressure changes that can mask or mimic changes in physiological pressure. As such, local atmospheric pressure changes can affect the physiological pressure measurement to a degree that the measured pressure data can become insufficiently precise for use in some medical applications.

### SUMMARY

One aspect of the invention relates to pressure measurement systems for determining a local atmospheric pressure and for determining a target physiological pressure that is corrected based on the local atmospheric pressure. In one embodiment, the pressure measurement system includes an implantable target pressure sensor, an implantable internal reference pressure sensor located remotely from the target pressure sensor, an external reference pressure sensor configured to transmit a local atmospheric pressure signal, and at least one condition indicator. The pressure measurement system further includes a controller configured to determine a correlation factor based on a signal from the internal reference pressure sensor and the signal from the external reference pressure sensor. The correlation factor can be determined at times when a telemetric transmission is received from the external reference pressure sensor. The controller is also configured to determine a calibrated reference pressure based on a signal from the internal reference pressure sensor, and also based on a stored correlation factor. This calibrated reference pressure can be determined at times when a telemetric transmission is not received from the external reference pressure sensor. Furthermore, at times when a telemetric transmission is received from the external reference pressure sensor, the controller can be configured to determine a relative physiological target pressure value by adjusting the signal from the target pressure sensor based on the signal from the external reference pressure sensor. At other times, the controller can be configured to adjust the signal from the target pressure sensor using a calibrated reference pressure.

Another aspect of the invention relates to methods for determining local atmospheric pressure from within the body of a patient. In one embodiment, the method includes providing an implantable local pressure sensor and at least one condition indicator within a human body, providing an implantable medical device having a controller that is configured to receive signals from the local pressure sensor and the at least one condition indicator, and providing an external reference pressure sensor that is configured to transmit a telemetric signal to the controller. The method can further include determining and storing a correlation factor when the external reference pressure sensor is in communication with the controller. The correlation factor can be based on a comparison of the signal from the implantable local pressure sensor and the signal from the external reference pressure sensor. The method can further include determining a local atmospheric pressure value. At times when the external reference pressure sensor is in communication with the controller, the local atmospheric pressure can be based on the signal received from the external reference pressure sensor. At other times when the external reference pressure sensor is not in communication with the controller, the local atmospheric pressure is determined by applying a stored correlation factor to the signal from the internal reference pressure sensor.

The invention may be more completely understood by considering the detailed description of various embodiments of the invention that follows in connection with the accompanying drawings. While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a medical device system in accordance with an embodiment of the invention.

FIG. 2 is a schematic view of several illustrative components of a controller in accordance with an embodiment of the invention.

FIG. 3 is a schematic view of a medical device system in accordance with another embodiment of the invention.

FIG. 4 is a schematic view of a medical device system in accordance with another embodiment of the invention.

FIG. 5 is a schematic view of a medical device system in accordance with another embodiment of the invention.

FIG. 6 is a schematic view of an implantable medical device in accordance with an embodiment of the invention.

FIG. 7 is a schematic view of an implantable medical device in accordance with an embodiment of the invention.

FIG. 8 is a partial cross-sectional schematic view of a portion of an implantable medical device in accordance with an embodiment of the invention.

FIG. 9 is a partial cross-sectional schematic view of a portion of an implantable medical device in accordance with another embodiment of the invention.

FIGS. 10A-10B are flow charts showing the operation of an implantable medical device system in accordance with an embodiment of the invention.

FIG. 11 is an example of a calibration matrix for use with embodiments of the invention.

While the invention may be modified in many ways, specifics have been shown by way of example in the drawings and will be described in detail. It should be understood, however, that the intention is not to limit the invention to the particular embodiments described. On the contrary, the intention is to cover all modifications, equivalents, and alternatives falling within the scope and spirit of the invention as defined by the claims.

### DETAILED DESCRIPTION

As discussed above, in cases where an implantable medical device is configured to measure an intracorporeal pressure, such as an intracardiac pressure, it may also be necessary to measure a reference pressure in order to account for changes in local atmospheric pressure. If changes in the local atmospheric pressure are not accounted for, these changes could render the intracorporeal target pressures insufficiently accurate for use in determining the patient's medical condition and for determining an appropriate medical therapy.

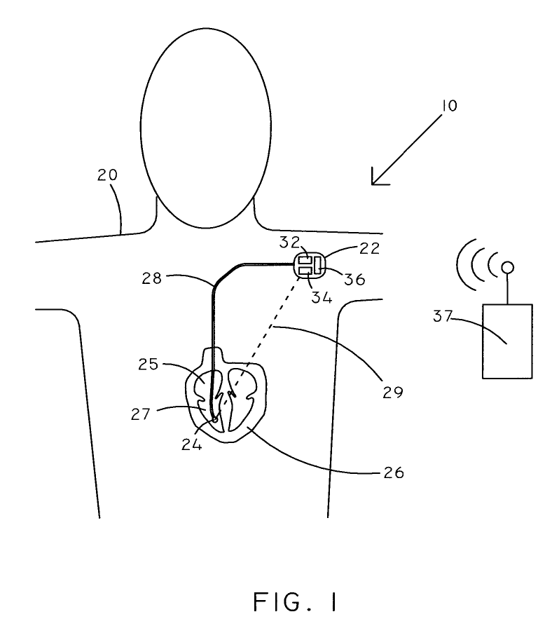

Embodiments of the invention include systems, devices, and methods for measuring intracorporeal pressure while accounting for changes in local atmospheric pressure. A medical device system according to an illustrative embodiment of the present invention is depicted in FIG. 1. The medical device system 10 is shown as implanted within a patient 20. System 10 includes an implantable target pressure sensor 24. The implantable target pressure sensor 24 can be configured to measure pressure within an intracorporeal area of interest (a target pressure). In the embodiment depicted in FIG. 1, the implantable target pressure sensor 24 is configured to detect pressure within a ventricle 27 of heart 26. In other embodiments, the implantable target pressure sensor 24 can be configured to sense pressures in other areas, such as the pressure within an atrium 25 of heart 26 or within an artery near heart 26, such as the pulmonary artery (not shown). The implantable target pressure sensor 24 can generate a signal that is representative of the absolute pressure that exists at the location of target pressure sensor 24, such as within a ventricle or atrium of the heart 26.

The target pressure sensor 24 can include any type of pressure sensor, for example an electrical, mechanical, or optical pressure sensor, that generates a signal in response to pressure. By way of example, exemplary pressure sensors are described in U.S. Pat. No. 6,237,398, the contents of which are herein incorporated by reference. The target pressure sensor 24 can be chronically implanted. The term “chronically implanted” as used herein with respect to a medical device shall refer to those medical devices that are implanted within an organism that are intended to remain implanted long-term, such as for a period of time lasting for months or years.

In an embodiment, the signal from the target pressure sensor 24 can be transmitted through a lead 28 to an implantable medical device 22. Lead 28 can pass from the heart 26 and through vasculature, such as the subclavian vein, and connect to the implantable medical device 22. Implantable medical device 22 can include a controller 32. The signal generated by target pressure sensor 24 can be received in the circuitry of controller 32. In another embodiment, the signal from target pressure sensor 24 can be transmitted wirelessly through a tissue path 29 and can be received at controller 32 by a wireless communications interface module.

Medical device system 10 further includes an implantable internal reference pressure sensor 34. The internal reference pressure sensor 34 can be configured to measure a pressure from within the body that reflects, or is correlated to, the local atmospheric pressure (i.e., an internal reference pressure). In the embodiment of FIG. 1, internal reference pressure sensor 34 is located near the controller 32. For example, internal reference pressure sensor 34 can be located in the same pocket formed in the patient's body tissue used to place implantable medical device 22. Alternatively, internal reference pressure sensor 34 may be located a distance away from controller 32 and connected to controller 32 by way of an additional lead or via a wireless connection.

Although it will be appreciated that the internal reference pressure sensor 34 can be located virtually anywhere within the body, in many embodiments the internal reference pressure sensor 34 is positioned within the patient's body at a location exhibiting a pressure that approximates or correlates to atmospheric pressure, such as within loose, fatty tissue near the skin surface. In some embodiments, the internal reference pressure sensor 34 is positioned within the patient's body at a location that is minimally influenced by muscle movements. For example, the internal reference pressure sensor 34 can be positioned away from tissue that is subject to pressure oscillations from respiration, cardiac contraction, or skeletal muscle contraction.

Similar to the target pressure sensor 24, the internal reference pressure sensor 34 can include any type of pressure sensor, for example an electrical, mechanical, or optical pressure sensor, that generates a signal in response to pressure. Exemplary pressure sensors are described in U.S. Pat. No. 6,237,398, the contents of which are herein incorporated by reference. In some embodiments, the internal reference pressure sensor 34 can be chronically implanted within the body.

Internal reference pressure sensor 34 generates a signal that corresponds to a pressure within the body cavity or pocket in which it is located. As stated previously, the internal reference pressure sensor 34 is preferably located in a body tissue region that is minimally influenced by muscle movements. In this way, the signal from internal reference pressure sensor 34 will tend to be correlated to the local atmospheric pressure. In some cases, the signal from internal reference pressure sensor 34 will correspond closely and directly to local atmospheric pressure. In other cases, the signal from internal reference pressure sensor 34 will be related to the local atmospheric pressure by a constant offset. In some cases, the correlation of internal reference pressure sensor 34 to actual local atmospheric pressure will vary by an offset amount that depends on the patient's posture, physical activity, and/or pulmonary activity and the offset amount can be uniquely determined for each combination of posture, physical activity, and/or pulmonary activity. As described in greater detail below, in some embodiments these offsets for each combination of posture, physical activity, and/or pulmonary activity can be stored in a calibration table or matrix and then later used by the system 10 in order to correct the internal reference pressure signal values.

In some embodiments, differences between an internal reference pressure signal value and an actual atmospheric pressure can be assessed by comparing the internal reference pressure signal with the signal values from an external reference pressure sensor. As such, system 10 further includes an external reference pressure sensor 37 (or non-implanted pressure sensor). External reference pressure sensor 37 is configured to measure local atmospheric pressure and transmit a telemetric signal that is representative of the local atmospheric pressure. Controller 32 is configured to receive the telemetric signal transmitted from external reference pressure sensor 37.

In an embodiment, external reference pressure sensor 37 is configured to be a primarily stationary device and is not carried with a patient during normal daily activities. However, in some embodiments, external reference pressure sensor 37 is configured to be moved or relocated as necessary to be co-located with the patient during the times that the patient is expected to be at rest. External reference pressure sensor 37 may have a limited telemetry transmission range, such as less than 5 meters in some embodiments, or less than 10 meters in other embodiments, or less than 50 meters in further embodiments. External reference pressure sensor 37 can be any type of pressure sensor, for example an electrical, mechanical, or optical sensor, that generates a signal in response to pressure.

External reference pressure sensor 37 can be included as part of a patient management system, such as the LATITUDE® patient management system, commercially available from Boston Scientific Corporation, Natick, Mass. Aspects of an exemplary patient management system are described in U.S. Pat. No. 6,978,182, the contents of which are herein incorporated by reference.

Medical device system 10 further includes one or more condition indicators 36. Data generated by the condition indicators 36 can aid in determining how to correct an absolute pressure measurement, as taken by target pressure sensor 24, to control for local atmospheric pressure measurements, as taken by an internal reference sensor 34. In the embodiment of FIG. 1, the one or more condition indicators 36 are co-located with controller 32. However, in other embodiments the condition indicators 36 are located remotely from the controller 32.

There are many usable embodiments of a condition indicator 36. In one usable embodiment, a condition indicator 36 is a sensor. The sensor can be one of many different types. For example, the sensor can be configured to sense body posture, body motion (e.g., patient activity), sounds, pulmonary function, cardiac function, temperature, time of day, and the like. In a specific embodiment, the condition indicator 36 is a body posture sensor, such as an accelerometer, that is configured to provide a signal that is representative of the body posture of the patient. For example, an accelerometer can be configured to provide an output signal due to the force of gravity which has a polarity and magnitude dependent on the degree to which a sensitive axis is tilted forward or rearward from the direction of earth's gravity. As such, a body posture sensor can be configured to sense a range of angles with respect to the earth's gravity, varying between a zero degree angle associated with lying down and a 90 degree angle associated with standing erect. Other intermediate angles may correspond to other body postures, such as sitting in a reclining chair. In some embodiments, an accelerometer, such as a DC accelerometer, can be mounted on an IC chip and disposed within the housing of the implantable medical device 22.

In an embodiment, a condition indicator 36 is a body motion sensor, such as a three-axis accelerometer that is configured to generate a signal corresponding to the presence and degree of body motion of the patient. An exemplary three-axis accelerometer is described in U.S. Pat. No. 6,937,900, the contents of which are herein incorporated by reference. In one embodiment, a single accelerometer is configured to be both a body motion sensor and a body posture sensor.

In an embodiment, the condition indicator 36 is a clock that provides a signal corresponding to the local time of day. The clock can be a timing circuit mounted on an IC chip and disposed within the housing of the implantable medical device 22.

In an embodiment, the condition indicator 36 is a pulmonary function sensor that is configured to provide a signal representative of the pulmonary function of the patient, such as the ventilation rate or ventilation volumetric rate (e.g., minute ventilation). For example, the condition indicator 36 can be an impedance sensor that measures impedance across body tissue in the patient's chest region, and the output of this impedance sensor can be used to determine minute ventilation. An exemplary minute ventilation sensing device based on transthoracic impedance is described in U.S. Pat. No. 6,868,346, the contents of which are herein incorporated by reference.

Some embodiments of medical device system 10 include the use of multiple condition indicators 36. For example, one embodiment of medical device system 10 includes an accelerometer for determining a patient's activity level and posture, a minute ventilation sensor, and a time of day sensor.

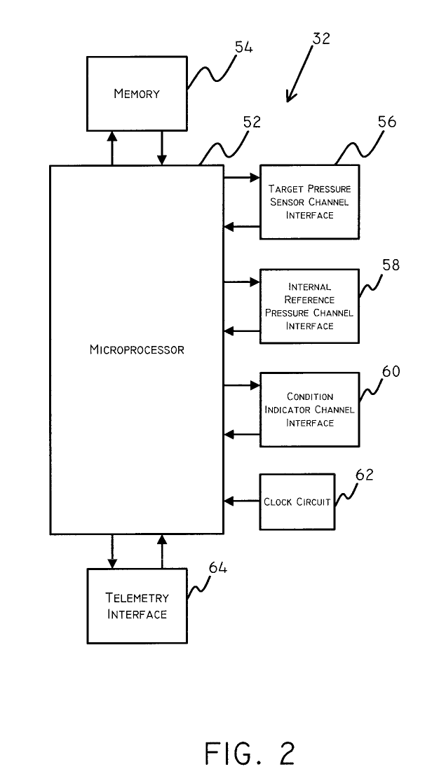

Controllers used in embodiments of the invention can include many different components in order to execute programs, store data, calculate values, and the like. Referring now to FIG. 2, some aspects of a controller 32 are schematically illustrated. In this embodiment, the controller 32 includes a microprocessor 52 that communicates with memory 54 via a bidirectional data bus. The memory 54 typically comprises ROM or RAM for program storage and a RAM for data storage. The controller 32 can include a bidirectional target pressure sensor channel interface 56 and a bidirectional internal reference pressure channel interface 58. The controller 32 can further include a condition indicator channel interface 60. The condition indicator channel interface 60 can be a conduit for signals from conditions indicators such as accelerometers, impedance sensors, minute ventilation sensors, activity sensors, and the like. In addition, the controller 32 can include a clock circuit 62. Finally, the controller 32 can include a telemetry interface module 64 for wireless communication of data into and out of the controller 32. For example, the telemetry interface module 64 can enable the controller 32 to receive an external reference pressure signal from the external reference pressure sensor 37.

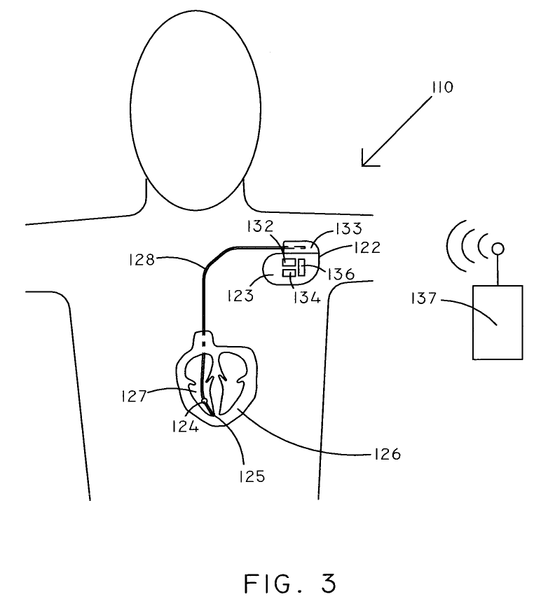

It will be appreciated that systems described herein can be associated with and/or include many different types of implantable medical devices. For example, systems described herein can be associated with or include features of pacemakers, implantable cardioverter-defibrillators (ICD), and the like. Referring now to FIG. 3, a schematic view of a medical device system 110 is shown in accordance with another embodiment of the invention. The implantable medical device 122 can be a pacemaker or an ICD. The implantable medical device 122 can include a housing 123 and a header 133. The implantable medical device 122 can include a controller 132. The controller 132 can be disposed within the housing 123. The controller 132 can be configured to initiate the delivery of electrical stimulation pulses to be delivered to a patient's cardiac tissue. The controller 132 can also be configured to execute various methods regarding the measurement of a physiological target pressure, such as those described in greater detail below. The implantable medical device 122 can include an internal reference pressure sensor 134, such as those described above. In an embodiment, the internal reference pressure sensor 134 can be disposed on or in the implantable medical device housing 123. The medical device system 110 can include one or more condition indicators 136. The medical device system 110 can also include an external reference pressure sensor 137, such as those described above. The implantable medical device 122 is connected to a lead 128. The lead 128 can provide electrical communication between the implantable medical device 122 and an electrode 125 disposed within a chamber of the heart 126, such as within the ventricle 127. In some embodiments, the medical device system 110 can include multiple leads and/or multiple electrodes. The implantable medical device 122 includes an implantable target pressure sensor 124. In certain embodiments, the implantable target pressure sensor 124 is disposed on the lead 128.

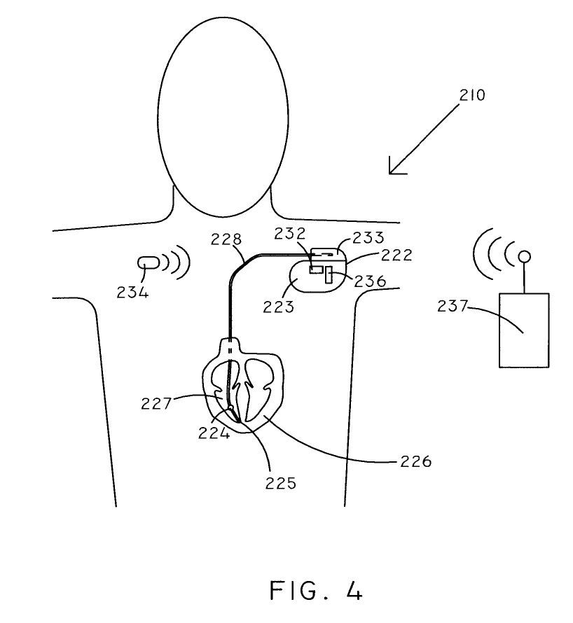

It will be appreciated that an internal reference pressure sensor can be located in many different places within the body. Referring now to FIG. 4, an embodiment of a medical device system 210 is shown including a remotely located internal reference pressure sensor 234. The internal reference pressure sensor 234 can be located virtually anywhere within the body. The internal reference pressure sensor 234 can be in wireless communication with an implantable medical device 222. The internal reference pressure sensor 234 can communicate with the implantable medical device 222 using various techniques including radiofrequency transmissions, acoustically, inductively, and the like. The implantable medical device 222 can include a housing 223 and a header 233. The implantable medical device 222 can include a controller 232 disposed within the housing 223. The medical device system 210 can include a condition indicator 236 disposed within the housing 223. The implantable medical device 222 is connected to a lead 228. The lead 228 provides electrical communication between the implantable medical device 222 and an electrode 225 disposed within a chamber of the heart 226, such as within the ventricle 227. The implantable medical device 222 includes an implantable target pressure sensor 224. The implantable target pressure sensor 224 can be configured to be disposed on the lead 228. The medical device system 210 can also include an external reference pressure sensor 237, such as those described above.

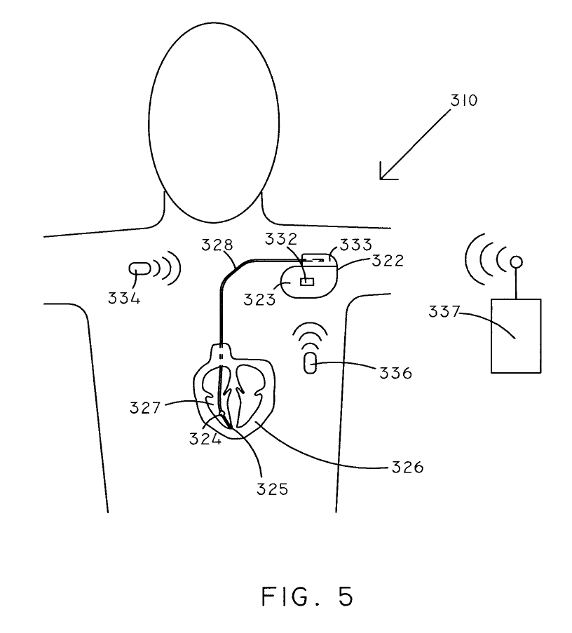

It will be appreciated that the one or more condition indicators can be located in many different places within the body. In many embodiments, the condition indicators are co-located with various components of the medical device system. However, referring now to FIG. 5, an embodiment of a medical device system 310 is shown including a remotely located condition indicator 336. The condition indicator 336 can include various types of sensors such as an activity sensor, an accelerometer, a pulmonary function sensor, and the like. In some embodiments, multiple condition indicators are positioned remotely from the other system components. The implantable medical device 322 can include a housing 323 and a header 333. The implantable medical device 322 can include a controller 332 disposed within the housing 323. The medical device system 310 can include an internal pressure sensor 334. The medical device system 310 can also include an external reference pressure sensor 337, such as those described above. The internal reference pressure sensor 334, the condition indicator 336, and the external reference pressure sensor 337 can all be in wireless communication with the implantable medical device 322. The internal reference pressure sensor 334, the condition indicator 336, and the external reference pressure sensor 337 can communicate with the implantable medical device 322 using various techniques including radiofrequency transmissions, acoustically, inductively, and the like. The implantable medical device 322 can be connected to a lead 328. The lead 328 provides electrical communication between the implantable medical device 322 and an electrode 325 disposed within a chamber of the heart 326, such as within the ventricle 327. The implantable medical device 322 includes an implantable target pressure sensor 324. In some embodiments, the implantable target pressure sensor 324 is disposed on the lead 328.

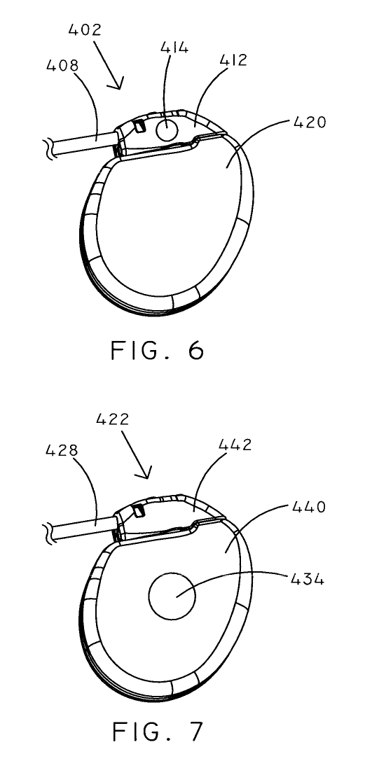

In some embodiments, the internal reference pressure sensor can be co-located with an implantable medical device. Referring now to FIG. 6, a schematic view of an implantable medical device 402 is shown in accordance with an embodiment of the invention. The implantable medical device 402 includes a housing 420 (sometimes referred to as a can) and a header 412. The housing 420 can serve to provide a sealed enclosure around various components of the device such as a controller and related circuitry. A lead 408 can be connected into the header 412. The header 412 can provide an interface between the lead 408 and components inside of the housing 420. An internal reference pressure sensor 414 can be disposed on the header 412. In some embodiment, the internal reference pressure sensor 414 can be disposed within the header 412.

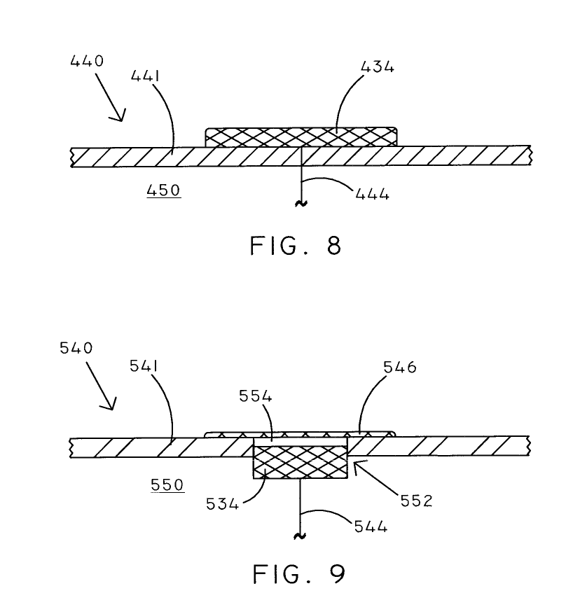

Referring now to FIG. 7, a schematic view of an implantable medical device 422 is shown in accordance with another embodiment of the invention. The implantable medical device 422 includes a housing 440 and a header 442. A lead 428 can be connected into the header 442. In this embodiment, an internal reference pressure sensor 434 is disposed on the housing 440. FIG. 8 shows a partial cross-sectional schematic view of a portion of the housing 440 in accordance with an embodiment of the invention. The housing 440 can include a housing wall 441. The housing wall 441 can be made of a material such as titanium, or other metals, polymers, or ceramics. The housing wall 441 can be configured to render the interior 450 of the housing 440 hermetically sealed. An internal reference pressure sensor 434 can be disposed on the surface of the housing wall 441. A conductor 444 can be configured to pass through the housing wall 441 and provide electrical communication between the internal reference pressure sensor 434 and components on the interior 450 of the housing wall 441, such as a controller. In other embodiments, communications between the internal reference pressure sensor 434 and components on the interior 450 of the housing wall 441 can be accomplished wirelessly.

In some embodiments, the internal reference pressure sensor can be disposed within the housing of an implantable medical device. FIG. 9 shows a partial cross-sectional schematic view of a portion of a housing 540 in accordance with another embodiment of the invention. The housing 540 can include a housing wall 541. The housing wall 541 can be made of a material such as titanium, or other metals, polymers, or ceramics. An internal reference pressure sensor 534 can be disposed within an aperture 552 in the housing wall 541. A membrane 546 can be disposed over the aperture 552 in the housing wall 541. In some embodiments, there can be a air gap 554, or channel, between the membrane 546 and the internal reference pressure sensor 534. The membrane 546 can be configured to allow pressure variations outside of the housing wall 541 to be sensed by the internal reference pressure sensor 534, while also preventing bodily fluids and tissues from entering into the interior 550. In some embodiments, the membrane 546 can include a polymeric material. For example, in some embodiments, the membrane 546 can include polytetrafluoroethylene (PTFE). In some embodiments, the membrane 546 can include a metal. For example, in some embodiments, the membrane 546 can include a flexible metal foil. A conductor 544 can be configured to provide electrical communication between the internal reference pressure sensor 534 and components on the interior 550 of the housing wall 541, such as a controller.

Embodiments of the invention can include various methods for measuring an intracorporeal pressure and devices and systems configured to execute the same. In some embodiments, methods can include procedures such as generating a pressure signal from a target physiological pressure sensor, generating an internal reference pressure signal, and generating signals from one or more condition indicators. Methods can also include determining if the patient is in an appropriate condition for taking physiological pressure measurements by evaluating the condition indicator signals. Methods can also include using an external reference pressure signal, if available, to generate corrected target pressure sensor signal values and to populate a calibration matrix. In some embodiments, if an external reference pressure signal is not available, then previously stored values in the calibration matrix can be used along with an internal reference pressure signal to generate corrected target pressure sensor signal values.

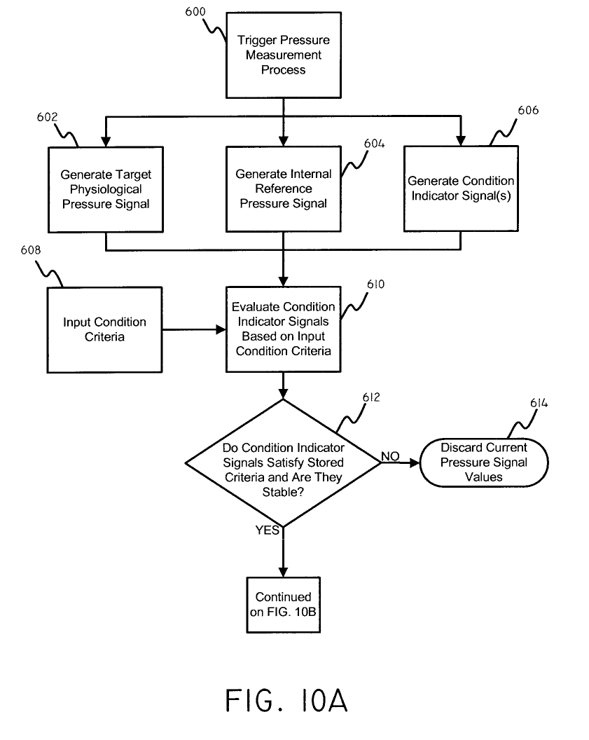

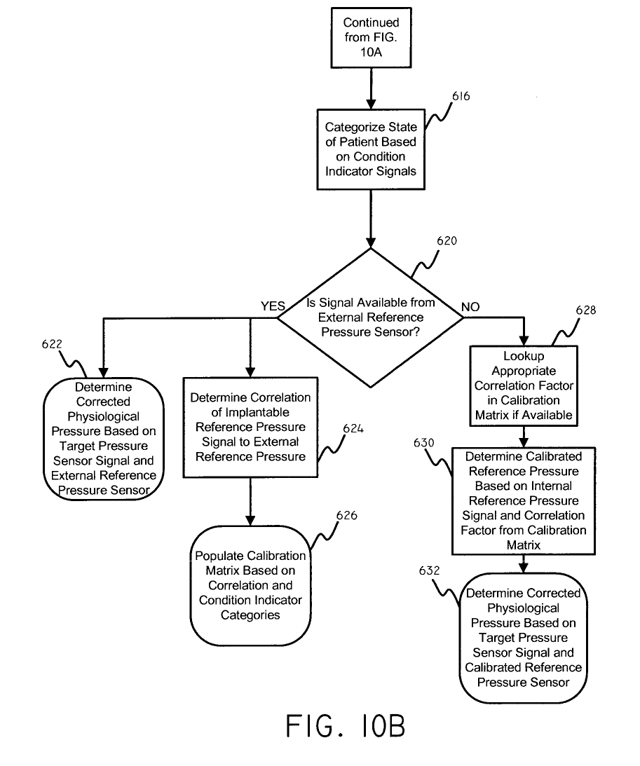

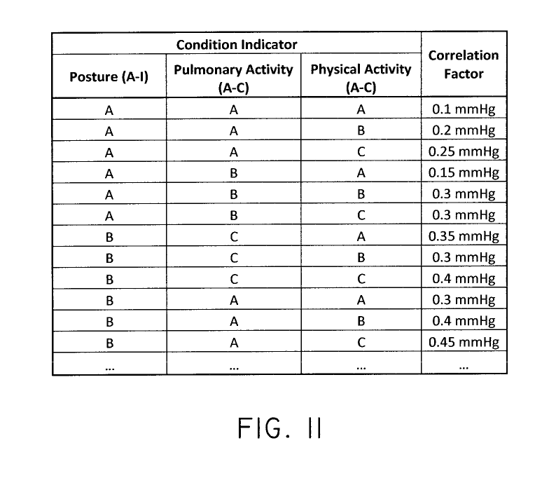

One example of various operations of a pressure measurement system in accordance with the present disclosure is illustrated in FIGS. 10A-10B. First, the pressure measurement process is triggered at step 600. The pressure measurement process may be triggered in various ways and at various time points. For example, in some embodiments, the pressure measurement process can be triggered by an external telemetric signal, such as a signal from an external patient management device. In some embodiments, the pressure measurement process is triggered automatically at various intervals of time. In some embodiments, the pressure measurement process can be automatically triggered during certain windows of time, such as between 12:00 AM and 4:00 AM. In some embodiments, the circumstances and timing of triggering the measurement process can be programmed into the system by a care provider.

Operation of a pressure measurement system can further include a step 602 of generating a signal from a physiological target pressure sensor, a step 604 of generating a signal from an internal reference pressure sensor, and a step 606 of generating a signal(s) from one or more condition indicators. By way of example, the one or more condition indicators can include an accelerometer for sensing the patient's posture, an accelerometer for sensing the patient's activity, a pulmonary function sensor, and/or a time of day clock.

In some embodiments, the controller can be configured to sample the signal values from the condition indicators a number of times to assess the rate of change of the signal values. In general, relatively constant condition indictor signals can be reflective of a desirable time to take pressure measurements. In some embodiments, condition indicator signal values are averaged over a period of time, such as 3 seconds, 5 seconds, or 10 seconds. Other periods of time are usable. Averaging the condition indicator measurements over a relatively short period of time helps to provide more accurate measurements that are less influenced by slight measurement variability. In some embodiments, a condition indicator signal is taken immediately before a pressure measurement and compared with a condition indicator signal value taken immediately after a pressure measurement. If the measurements change more than a predetermined amount, then the controller can repeat the measurement process and delay further processing until there is consistency and stability in the measurements. Alternatively, if the measurements change more than a predetermined amount, the measurement process can be terminated until it is triggered again.

At step 608, condition criteria can be input into the system and stored in memory. Step 608 can be conducted at various points in time. For example, step 608 can be done when the system is initially implanted into a patient. Alternatively, step 608 can be done by a care provider via a programmer during a follow-up visit. Each criterion can be associated with a particular condition indicator. Each criterion can represent a threshold value or range of values that represents a degree of the patient's condition, for example a degree of pulmonary exertion. The criteria can serve to distinguish between when pressure measurements are likely to be accurate and when pressure measurements are likely to be inaccurate because of factors such as the physical activity of the patient. For example, in the context of a condition indicator that is a pulmonary function sensor, then a criterion can reflect a threshold level of pulmonary exertion, above which pressure measurements should not be assessed because of possible inaccuracy stemming from the degree the pulmonary exertion. Each condition indicator can have its own criteria.

At step 610, the condition indicator signals are evaluated to identify times when it is appropriate to take pressure measurements. For example, criteria can be applied to the condition indicator signals, as discussed above, in order to identify times when the patient is in a generally sedentary state, where pressure measurements will not be unduly influenced by the patient's body motion. Specifically, the controller can compare the signals from the condition indicators to the associated criteria. This comparison can be performed by a processor associated with the controller. The scope of the comparison step can depend on the number of condition indicators present and the number of criteria for each condition indicator.

If the criteria are not satisfied or if the condition indicator signals are not sufficiently stable, then at step 614 the current pressure signal values are discarded and a corrected target physiological pressure is not determined. In some embodiments, the controller can continue to receive signals from the condition indicators until the criteria are satisfied. In some embodiments, the system ceases operations until the process is triggered again.

If the criteria are satisfied by the signal from the respective condition indicator and if the condition indicator signals are sufficiently stable, then at step 616 (see FIG. 10B) the signals from the condition indicator signal values are categorized according to the specific value of each condition indicator signal. Categorizing the condition indicator signals can be advantageous because it allows the system to more accurately correct the target physiological pressure. The specific number of categories that each signal value is placed into can be configured as desired. In one embodiment, the number of categories for a given condition indicator signal is a programmable value that can be entered or modified by a user, such as a care provider.

As a specific example of a categorization process, the signal from a posture sensor (an exemplary condition indicator) can be categorized into one of nine categories, where each category represents a 10 degree interval in the range between 0 degrees (laying flat) and 90 degrees (standing erect). For purposes of illustration herein, these categories may be arbitrarily labeled “A” for the range 0-10 degrees, “B” for the range 11-20 degrees, and so forth, up to “I” for the range 81-90 degrees. While nine categories are illustrated for a signal from a posture sensor here, it will be appreciated that other specific numbers of categories can be used. For example, a user can select that three or six, or any other desired number of categories are to be used. Signals from other types of condition indicators can be similarly categorized. For example, in an embodiment, the signal from an activity sensor can be categorized into a specific number of categories, such as three categories, wherein a first category “A” represents a relatively low activity level, a second category “B” represents a relatively moderate activity level, and a third category “C” represents a relatively high activity level. In some embodiments, these activity level categories all represent a degree of activity below a threshold level as defined by an input criterion.

A signal from a condition indicator representing pulmonary function can be similarly categorized. For example, the signal can be categorized into one of three categories, where one category “A” represents a relatively low pulmonary activity level, another category “B” represents a relatively moderate pulmonary activity level, and a third category “C” represents a relatively high pulmonary activity level. The number of different condition indicator signals that are categorized can be different in different embodiments. For example, in one embodiment, the only categorization may be of a condition indicator signal representing the patient's posture.

Because an external reference pressure sensor may be disposed in one particular physical location and has a limited range of wireless communication, a pressure measurement system as described herein and implanted in a patient will sometimes be in communication with the external reference pressure sensor and sometimes not be in communication with the external reference pressure sensor. At step 620 the controller assesses whether a signal is available from an external reference pressure sensor. If a signal is found to be present, such as where the patient is located within the wireless transmission range of the external reference pressure sensor, the signal can be used to adjust the target pressure measurement sensor signal for atmospheric pressure at step 622. For example, the target pressure sensor signal can be adjusted based on the measurement from the external reference pressure sensor to yield a corrected physiological pressure measurement.

In addition, where a signal from an external reference pressure sensor is available, the system can use that signal to determine the difference between the external reference pressure sensor and the internal reference pressure sensor at step 624. At step 626, this differential value can be used to populate a calibration matrix. The calibration matrix can be a table stored in memory or a register that contains unique values for different possible combinations of condition indicator categorizations. The calibration matrix can have a total number of entries that depends on the number of condition indicator signals used and the number of categories for each condition indicator signal. For example, where three different condition indicator signals are used (such as pulmonary activity, physical activity, and posture) and where pulmonary activity and physical activity can each be categorized into three different categories and where posture can be categorized into nine different categories, then the total number of differential values (or cells) in the calibration matrix can be equal to 3×3×9, or 81. Each type of condition indicator signal can be referred to as a dimension of the calibration matrix. For example, a calibration matrix that accounts for categories of pulmonary activity, physical activity, and posture can be referred to as a three-dimensional calibration matrix.

Referring to FIG. 11, an illustration of a portion of a hypothetical three-dimensional calibration matrix is shown. The calibration matrix is three-dimensional and includes nine categories for posture (A-I), three categories for pulmonary activity (A-C), and three categories for physical activity (A-C). For each possible unique categorization, a correlation factor can be provided. Though the correlation factors are shown in units of mmHg, it will be appreciated that they can also be stored in a calibration matrix in other units as well.

In operation, after the correlation factor between the internal reference pressure sensor and the external reference pressure sensor is determined, this pressure differential can used at step 626 to populate the cells of the calibration matrix. Population of the calibration matrix can be performed in various ways. In an embodiment, the most recent pressure differential value for one particular cell (corresponding to a particular combination of pulmonary activity, physical activity, posture) is stored, replacing all previous values. In another embodiment, the most recently determined pressure differential value is averaged with previously stored differential values, or in other embodiments, with some portion of the previously stored differential values, such as those measurements taken within a certain time period. In some embodiments, the system continues to populate the calibration matrix as long as the external reference pressure signal is received by the implanted medical device.

Where a signal from an external reference pressure sensor is not available, the system can still proceed to calculate a corrected physiological target pressure. Specifically, at step 628, the system can lookup an appropriate correlation factor from a calibration matrix, if the appropriate cell of the calibration matrix has previously been populated with a correlation factor. If the appropriate cell of the calibration matrix has not yet been populated, then the current pressure measurements can simply be discarded and a corrected target physiological pressure is not determined. However, where an appropriate cell of the calibration matrix has been populated, then this correlation factor can be used to determine a calibrated internal reference pressure based on the signal from the internal reference pressure sensor at step 630. As an illustration, where the internal reference pressure sensor indicates a value of 765 mmHg, and the calibration matrix indicates a differential of 0.4 mmHg for the specific categorization, then the calibrated reference pressure signal value can be taken as 765.4 mmHg. Next, the calibrated reference pressure value can be used in conjunction with the target pressure sensor signal in order to determine a corrected physiological pressure at step 632. For example, the calibrated reference pressure value can be subtracted from the target pressure sensor signal value.

Corrected physiological target pressure values can be used to aid in the diagnosis and/or monitoring of various conditions. In some embodiments, after a corrected physiological target pressure is determined, whether at step 622 or at step 632, the value can be stored in the memory of the controller. In this way, the current physiological target pressure value can be compared with future physiological target pressure values in order to identify changes that may be meaningful. In some embodiments, the controller also stores a time stamp with each stored corrected physiological pressure value. In some embodiments, the controller can be configured to use the stored physiological target pressure values to determine a therapy to administer, or can be configured to transmit the stored data to an external device for analysis and review.

In some embodiments, the calibration matrix can be two dimensional or even one-dimensional. For example, a two dimensional calibration matrix may be based only on posture category and pulmonary activity. An exemplary one dimensional calibration matrix is one based only on the posture category.

It will be appreciated that the steps shown in FIGS. 10A and 10B are provided only by way of illustration and that in some embodiments, some of the steps can be performed in a different order than is shown. In addition, in some embodiments, some of the steps can be omitted.

In some embodiments, the step of determining a correlation of an implantable reference pressure sensor to an external reference pressure sensor can involve determining coefficients of an equation based on the difference between the sensor signals, where the equation may be a linear equation or a non-linear equation that is used to correct the implanted sensor data to the non-implanted sensor data.

In various embodiments, the process of generating the signals from the internal sensors and condition indicators is substantially continuous. In some embodiments, after the controller has completed one of steps 614, 622, or 632, the process can be repeated again immediately. In this way, changes to the patient's medical condition can be determined in real time. However, in other embodiments the process is repeated only periodically.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the present specification. The claims are intended to cover such modifications and devices.

The above specification provides a complete description of the structure and use of the invention. Since many of the embodiments of the invention can be made without departing from the spirit and scope of the invention, the invention resides in the claims.

What is claimed is:

1. A pressure measurement system, comprising:
an implantable target pressure sensor;
an implantable internal reference pressure sensor located remotely from the target pressure sensor;
an external reference pressure sensor configured to wirelessly transmit a local atmospheric pressure signal; and
a controller in communication with the implantable target pressure sensor and the implantable internal reference pressure sensor, the controller configured to:
determine a correlation factor between a signal from the implantable internal reference pressure sensor and the local atmospheric pressure signal from the external reference pressure sensor at times when the external reference pressure sensor is in communication with the controller;
determine a calibrated reference pressure based on a signal from the implantable internal reference pressure sensor and a correlation factor at times when the external reference pressure sensor is not in communication with the controller; and
determine a relative physiological target pressure value
at times when the external reference pressure sensor is in communication with the controller, by adjusting the signal from the target pressure sensor by the signal from the external reference pressure sensor; and
at times when the external reference pressure sensor is not in communication with the controller, by adjusting the signal from the target pressure sensor by a calibrated reference pressure.

2. The pressure measurement system of claim 1, further including at least one condition indicator in communication with the controller.

3. The pressure measurement system of claim 2, wherein the at least one condition indicator includes a body motion sensor.

4. The pressure measurement system of claim 2, wherein the at least one condition indicator includes a body posture sensor.

5. The pressure measurement system of claim 2, wherein the at least one condition indicator includes a time of day sensor.

6. The pressure measurement system of claim 2, wherein the at least one condition indicator includes a pulmonary function sensor.

7. The pressure measurement system of claim 1, wherein the controller is configured to determine a correlation factor between a signal from the implantable internal reference pressure sensor and the local atmospheric pressure signal from the external reference pressure sensor by subtracting the value of the external reference pressure sensor from the value of the implantable internal reference pressure sensor.

8. The pressure measurement system of claim 1, wherein the correlation factor is stored in a calibration matrix.

9. The pressure measurement system of claim 2, wherein the controller is configured to categorize a state of a patient based on a signal from the at least one condition indicator.

10. The pressure measurement system of claim 2, wherein the controller is configured to evaluate the rate of change of a signal from the at least one condition indicator.

11. A pressure measurement system comprising:
an implantable target pressure sensor;
an implantable internal reference pressure sensor located remotely from the target pressure sensor;
an external reference pressure sensor configured to wirelessly transmit a local atmospheric pressure signal;
at least one condition indicator; and
a controller in communication with the implantable target pressure sensor, the implantable internal reference pressure sensor, and the at least one condition indicator, the controller configured to:
determine a correlation factor between a signal from the implantable internal reference pressure sensor and the local atmospheric pressure signal from the external reference pressure sensor at times when the external reference pressure sensor is in communication with the controller;
determine a calibrated reference pressure based on a signal from the implantable internal reference pressure sensor and a correlation factor at times when the external reference pressure sensor is not in communication with the controller; and
determine a relative physiological target pressure value in response to a signal from the at least one condition indicator.

12. A method for determining local atmospheric pressure from within the body of a patient, the method comprising:
providing an implantable local pressure sensor and at least one condition indicator within a human body;
providing an implantable medical device comprising a controller that is configured to receive signals from the implantable local pressure sensor and the at least one condition indicator;
providing an external reference pressure sensor that is configured to transmit a wireless signal to the controller;
determining and storing a correlation factor when the external reference pressure sensor is in communication with the controller; the correlation factor based on a comparison of a signal from the implantable local pressure sensor and the signal from the external reference pressure sensor; and
determining a local atmospheric pressure value.

13. The method of claim 12, wherein determining a local atmospheric pressure value is based on a signal from the external reference pressure sensor when the external reference pressure sensor is in communication with the controller.

14. The method of claim 12, wherein determining a local atmospheric pressure value is based on a signal from the implantable local pressure sensor and a stored correlation factor when the external reference pressure sensor is not in communication with the controller.

15. The method of claim 12, wherein the at least one condition indicator includes a body motion sensor.

16. The method of claim 12, wherein the at least one condition indicator includes a body posture sensor.

17. The method of claim 12, wherein the at least one condition indicator includes a time of day sensor.

18. The method of claim 12, further comprising:
providing an implantable target pressure sensor; and
determining a relative target pressure based on a signal from the implantable target pressure sensor and the determined local atmospheric pressure value.

19. The method of claim 12, further comprising selecting a stored correlation factor from a calibration matrix based on values of signals from the at least one condition indicator.

20. The method of claim 12, further comprising categorizing a state of the patient based on a signal from the at least one condition indicator.
